(19) United States
(12) Patent Application Publication
(10) Pub. No.: US 2024/0158763 A1
(43) Pub. Date: May 16, 2024

(54) OPTIMAL PRODUCTION OF SARS-COV-2 VIRUS-LIKE PARTICLES (VLPS) PRODUCED IN MAMMALIAN CELLS

(71) Applicant: Arizona Board of Regents on Behalf of Arizona State University, Scottsdale, AZ (US)

(72) Inventor: Brenda HOGUE, Tempe, AZ (US)

(21) Appl. No.: 18/552,762

(22) PCT Filed: Mar. 30, 2022
(86) PCT No.: PCT/US2022/022547

§ 371 Date: Sep. 27, 2023

Related U.S. Application Data
(60) Provisional application No. 63/168,148, filed on Mar. 30, 2021.

(51) Int. Cl. C12N 7/00; A61K 39/00; A61K 39/12; A61P 31/14; C12N 15/85

(52) U.S. Cl. CPC C12N 7/00; A61K 39/12; A61P 31/14; C12N 15/85; A61K 2039/5258; A61K 2039/543; A61K 2039/55516; A61K 2039/55577; C12N 2770/20023; C12N 2770/20034; C12N 2770/20051

(57) ABSTRACT

Severe acute respiratory syndrome coronavirus 2 (SARS-CoV-2) virus-like particles (VLP) are provided herein as well as methods of making and using the same. The methods of making the VLPs include transfecting a mammalian cell with expression vectors allowing for expression of at least the SARS-CoV-2 M, E and S proteins to make the VLPs. The VLPs can be administered to a subject to induce an immune response against SARS-CoV-2.

### CROSS-REFERENCE TO RELATED APPLICATIONS

This patent application claims the benefit of priority of U.S. Provisional Patent Application No. 63/168,148 filed on Mar. 30, 2021, which is incorporated herein by reference in its entirety.

### SEQUENCE LISTING

This application is being filed electronically via EFS-Web and includes an electronically submitted Sequence Listing in .txt format. The .txt file contains a sequence listing entitled “112624_01331_ST25.txt” created on Mar. 30, 2022 and is 75,207 bytes in size. The Sequence Listing contained in this .txt file is part of the specification and is hereby incorporated by reference herein in its entirety.

### BACKGROUND

Coronaviruses (CoV) constitute a large family of positive-stranded, enveloped RNA viruses that infect a broad range of mammalian and avian species. The viruses cause primarily respiratory and enteric diseases. In the last two decades three new zoonotic CoVs have emerged to infect humans. The most recent emergence of SARS-CoV-2 that continues to spread globally raises many scientific and public health questions and challenges. Development of effective vaccines and antiviral therapeutics and rapidly deployment of both is a pressing need. This will be an even more critical priority if SARS-CoV-2 continues to spread and becomes endemic in the respiratory virus disease landscape. Previous work with the other two recent emergent pathogenic human CoVs, severe acute respiratory syndrome (SARS-CoV) and Middle East respiratory syndrome (MERS-CoV), provides insight and platforms that can help expedite the process, but none of these have moved beyond early trial stages. Much remains to be learned about the SARS-CoV-2 and its interplay with its human host and what will constitute the most effective, safe vaccine strategy. There are currently seven CoVs that infect humans, HCoVs OC43, 229E, NL63 and HKU1, that cause seasonal upper respiratory infections, in addition to the three more pathogenic viruses. The human viruses are thought to have emerged from zoonotic hosts to infect humans. Viral genomic analyses indicate that the human viruses are related to bat CoVs. A large number of novel CoVs have been identified in bat populations since identification of SARS-CoV and the expectation is that we will continue to have spillover of these viruses to humans. This reinforces the need for development of vaccines against emergent CoVs.

### SUMMARY OF THE INVENTION

In a first aspect, provided herein is a Severe Acute Respiratory Syndrome Coronovirus 2 (SARS-CoV-2) virus-like particle (VLP) comprising SARS-CoV-2 spike (S) protein (SEQ ID NO:1) or a sequence at least 95% identical thereto, SARS-CoV-2 membrane (M) protein (SEQID NO:7) or a sequence at least 95% identical thereto, SARS-CoV-2 envelope (E) protein (SEQ ID NO:8) or a sequence at least 95% identical thereto, and lacking SARS-CoV-2 viral genome. In some embodiments, the VLP additionally comprises SARS-CoV-2 nuceocapsid (N) protein (SEQ ID NO:9) or a sequence at least 95% identical thereto.

In a second aspect, provided herein is a method for producing a SARS-CoV-2 VLP described herein comprising transfecting a mammalian cell with (i) a first polynucleotide encoding SARS-CoV-2 S protein (SEQ ID NO:1) or a sequence at least 95% identical thereto; (ii) a second polynucleotide encoding SARS-CoV-2 M protein (SEQ ID NO:7) or a sequence at least 95% identical thereto; and (iii) a third polynucleotide encoding SARS-CoV-2 E protein or a sequence at least 95% identical thereto; and extracting the SARS-CoV-2 VLP from the mammalian cell. In some embodiments, the method additionally comprises transfecting the mammalian cell with (iv) a fourth polynucleotide encoding SARS-CoV-2 N protein (SEQ ID NO:9) or a sequence at least 95% identical thereto. In some embodiments, the first, second, third, and/or fourth polynucleotide is codon optimized for expression in a mammalian cell. In some embodiments, the first, second, and third polynucleotide are transfected into the mammalian cell using at least one vector. In some embodiments, a single vector comprises the first, second, and third polynucleotides. In some embodiments, the mammalian cell is transfected with a first vector comprising the first polynucleotide, a second vector comprising the second polynucleotide, and a third vector comprising the third polynucleotide. In some embodiments, the vector additionally comprises a promoter. In some embodiments, the promoter is a chicken β-actin promoter or a CMV promoter. In some embodiments, the mammalian cell is selected from the group consisting of Chinese Hamster Ovary (CHO), Madin-Darby Canine Kidney (MDCK), Vero and HEK 293T.

In a third aspect, provided herein is a vaccine composition comprising a SARS-CoV-2 VLP as described herein and a pharmaceutically acceptable carrier. In some embodiments, the vaccine additionally comprises an adjuvant. In some embodiments, the adjuvant is a mucosal adjuvant selected from the group consisting of LT mutant R1925G, saponin, and IVX-908 proteosome formulation.

In a fourth aspect, provided herein is a method for inducing an immune response in a host comprising administering an effective amount of a vaccine composition as described herein to the host. In some embodiments, the host is human. In some embodiments, the vaccine composition is administered systemically. In some embodiments, the vaccine composition is administered by injection. In some embodiments, the vaccine composition is administered intranasally.

### BRIEF DESCRIPTION OF THE DRAWINGS

FIG. 1 shows a schematic of a CoV virus, which comprises at least three envelope structural proteins, the membrane (M), spike (S), and envelope (E).

FIG. 2 shows a schematic representation of the SARS-CoV S protein. The approximate positions of the receptor binding domain (RBD), fusion peptide (FP), heptad repeats (HR), and transmembrane domains (TM) within the S1 and S2 regions are shown. The S protein of SARS-CoV-2 is structured similarly.

FIG. 3 shows expression of SARS-CoV-2 genes singly in the pCAGGS vector. Proteins were expressed following transfection into 293T cells (A). Protein expression in the intracellular cytoplasmic fractions (B) and extracellular media (C) were analyzed by SDS-PAGE and western blotting using SARS-2 protein specific antibodies. The S, M and N proteins are not released into the extracellular media when expressed alone (C, lanes S, M, N). The envelope (E) protein is released when expressed alone, (C, lane E). Co-expression of the M and E protein is the minimal requirement for envelope formation as shown by EM negative staining (D). The empty vector (V) control is shown in each panel.

FIG. 4 shows a vector map of the pCAGGS vector comprising the SARS-CoV-2 genes encoding the M, S, E, and nucleocapsid (N) proteins.

FIG. 5A-5D shows SARS-Co-V-2 virus like particles (VLPs). A) Proteins expressed in 293T cells following transfection. M-E indicated co-expression of the M-IRES-E from the same plasmid. B) Protein expression in the intracellular cytoplasmic fraction and VLPs in extracellular media analyzed by SDS-PAGE and Western blotting using SARS-2 protein specific antibodies. S, M and N proteins are not released into the extracellular medial when expressed singly, but M is present when co-expressed with E and M, indicative of VLP formation. C) TEM images of purified SARS-2 VLPs. D) Example of one multi-gene expression plasmid with all genes under the control of a CMV promoter.

FIG. 6 shows C57/B16 mice (10-12 weeks old) were inoculated intranasally with MA at indicated days post-infection. Weight loss (top) and survival (bottom) are shown.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION OF THE INVENTION

The present disclosure describes severe acute respiratory syndrome coronavirus 2 (SARS-CoV-2) vaccine compositions, as well as methods for making and using the same.

Severe acute respiratory syndrome coronavirus 2 (SARS-CoV-2) is the third highly pathogenic human CoV to emerge in the past two decades. The virus causes COVID-19, a severe respiratory disease with an estimated mortality of 2-3% that rapidly spread across China beginning in late 2019. The virus also spread globally to 29 countries during this period. Like other CoVs, the spike (S) protein is assumed to be the major target for neutralizing antibodies. SARS-CoV-2 S protein binds to the receptor, angiotensin-converting enzyme 2 (ACE2), through its receptor binding domain (RBD). The RBDs for other CoVs are immunogenic and a major neutralizing determinant. Significant research has been directed toward vaccine development for the other recently emerged human pathogenic CoVs, severe acute respiratory syndrome (SARS) and Middle East respiratory syndrome (MERS), but there is a need for additional vaccines. There are significant concerns about the continuing spread of the virus and the possibility that it will become embedded in the viral respiratory disease landscape that will be encountered seasonally. Thus, development of further safe and effective vaccines against the virus is a significant priority. The long-term goal of this project is to develop a safe, efficacious vaccine(s) against SARS-CoV-2. Standard molecular biology, biochemical approaches, vaccination and immunogenicity assessment will be used to generate virus-like-particles (VLPS) in mammalian cells. The goal of this work is to optimally produce VLPs in mammalian cells and evaluate immune responses elicited in mice vaccinated with the VLPS.

SARS-CoV-2 includes membrane (M), spike (S), envelope (E), and nucleocapsid (N) structural proteins. The M, S, and E proteins provide the structure of the exterior viral envelope.

The S protein is a glycoprotein that mediates receptor binding and fusion during entry into a host cell. The S protein of SARS-CoV-2 has the sequence of SEQ ID NO:1. The receptor binding domain (RBD, SEQ ID NO:6) is amino acids 318-510 of SEQ ID NO:1.

In some embodiments, the native S protein has been modified to improve its expression from the vaccines described herein. For example, the inventors have generated a modified S protein (SEQ ID NO:14) that includes a mutation in the furin cleavage site and several proline mutations to stabilize the pre-fusion S protein.

In some embodiments, the native RBD domain has been modified to reflect mutations found in variants of interest of SARS-CoV-2 (Table 1 and SEQ ID NOs 19-38).

TABLE 1
RBD Mutations

| | | | | | SARS2- |
|---|---|---|---|---|---|
| | Beta | Gamma | Delta | Omicron | N501Y_{MA30} |
| | (SEQ ID NO) | (SEQ ID NO) | (SEQ ID NO) | (SEQ ID NO) | (SEQ ID NO) |
| G339 | — | — | — | G339D (19) | — |
| S371 | — | — | — | S371L (20) | — |
| S373 | — | — | — | S373P (21) | — |
| S375 | — | — | — | S375F (22) | — |
| K417* | K417N (23) | K417T (24) | — | K417N (23) | K417M (25) |
| N440* | — | — | — | N440K (26) | — |
| G446 | — | — | — | G446S (27) | — |
| L452 | — | — | L452R (28) | — | — |
| S477 | — | — | — | S477N (29) | — |
| T478 | — | — | T478K (30) | T478K (30) | — |
| E484* | E484K (31) | E484K (31) | — | E484A (32) | E484K (31) |
| Q493* | — | — | — | Q493K (33) | Q493R (34) |
| G496 | — | — | — | G496S (35) | — |
| Q498 | — | — | — | Q498R (36) | Q498R (36) |
| N501* | N501Y (37) | N501Y (37) | — | N501Y (37) | N501Y (37) |
| Y505 | — | — | — | Y505H (38) | — |

*Mutations associated with antibody escape

Notably SEQ ID NO: 19-38 contain individual mutations identified in the S protein and these individual mutations may be combined to form novel S proteins that may be used to generate the vaccines and VLPs described herein. For example, the delta variant of the virus contains the mutations provided in SEQ ID NO: 28 and 30 in combination. Thus, these described combinations as well as new combinations are also provided herein. It is likely that new combinations of mutations in the S protein will continue to arise in the circulating virus population and the vaccines and VLPs described herein will need to take account of the circulating virus in order to maintain immunogenicity.

The M protein of SARS-CoV-2 has the sequence of SEQ ID NO:7. The E protein of SARS-CoV-2 has the sequence of SEQ ID NO:8. The N protein is an internal structural component that encapsulates the SARS-CoV-2 viral genome. The N protein of SARS-CoV-2 has the sequence of SEQ ID NO:9.

As used herein, “SARS-CoV-2 antigen” refers to a SARS-CoV-2 protein, a sequence at least 90% identical thereto, a fragment thereof, or combinations thereof that may be used to elicit an immune response in a subject. The SARS-CoV-2 antigen may be the SARS-CoV-2 S protein, the SARS-CoV-2 M protein, the SARS-CoV-2 E protein, the SARS-CoV-2 N protein, the SARS-CoV-2 S protein RBD, a protein with a sequence at least 90%, 95%, 98%, or 99% sequence identity thereto, or combinations thereof.

In some embodiments, the DNA sequences of the SARS-CoV-2 antigens are codon-optimized for expression in mammals. For example, in some embodiments, the S protein has the sequence of SEQ ID NO:15, the E protein has the sequence of SEQ ID NO:16, the M protein has the sequence of SEQ ID NO:17, and the N protein has the sequence of SEQ ID NO:18.

As used herein, the phrases “% sequence identity,” “percent identity,” or “% identity” are used interchangeably and refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. A suite of commonly used and freely available sequence comparison algorithms is provided by the National Center for Biotechnology Information (NCBI) Basic Local Alignment Search Tool (BLAST® alignment tool), which is available from several sources, including the NCBI, Bethesda, Md., at its website. The BLAST® alignment tool software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Polynucleotides encoding any of the SARS-CoV-2 antigens described herein are provided. As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA or genomic DNA.

Polynucleotides homologous to the polynucleotides described herein are also provided. Those of skill in the art understand the degeneracy of the genetic code and that a variety of polynucleotides can encode the same polypeptide. In some embodiments, the polynucleotides (i.e., polynucleotides encoding the SARS-CoV-2 antigens described herein) may be codon-optimized for expression in a particular cell including, without limitation, a plant cell, mammalian cell, insect cell, bacterial cell, or fungal cell. While particular polynucleotide sequences are disclosed herein, any polynucleotide sequences may be used which encodes a desired form of the polypeptides described herein. Thus non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

In another aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a promoter operably linked to any one of the polynucleotides described herein. As used herein, a polynucleotide is “operably connected” or “operably linked” when it is placed into a functional relationship with a second polynucleotide sequence.

As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. Suitable promoters are known and described in the art. Other promoters include herpes simplex virus thymidine kinase, adenovirus major late promoter, phosphoglycerate kinase, human elongation factor 1 alpha the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, mammalian synthetic promoter libraries, and the like as well as the translational elongation factor EF-1α promoter or ubiquitin promoter. In some embodiments, the promoter is viral synthetic late promoter (SLP). In some embodiments, the SLP has the sequence of SEQ ID NO:5. Those of skill in the art are familiar with a wide variety of additional promoters for use in various cell types.

In some embodiments, the constructs may include a selection marker. The selection marker may be used to monitor transfection efficiency or for selection of stable constructs. The selection marker may include bleomycin, Zeocin, adenosine or cytosine deaminase, aminoglycoside phosphotransferase, hygromycin, G418/Geneticin, GFP, m-Cherry or any other such marker known in the art.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Viral genomes are also included as vectors, including vectors based on viral genomes. Vectors may carry genetic elements, such as those that confer resistance to certain drugs or chemicals. Examples of vectors that may be used, include, but are not limited to VAMsyB and pCAGGS. A BacMam expression system that uses baculovirus to deliver genes into mammalian cells could also be used as an alternative.

In some embodiments, vectors described herein include an internal ribosomal entry site (IRES). In some embodiments, vectors described herein include at least two IRES. In some embodiments, vectors described herein include a self-cleaving protein element. In some embodiments, vectors described herein include at least two self-cleaving protein elements.

In some aspects, provided herein are virus-like particles (VLPs) or recombinant immune complexes incorporating the SARS-CoV-2 antigens described herein.

As used herein, “virus-like particles (VLPs)” refers to particles that include one or more viral proteins and mimics the structural of the native virus but lack the viral genome. In some embodiments, the VLP includes at least the S protein. In some embodiments, the VLP includes at least the M and E proteins. In some embodiments, the VLP includes at least the M, E, and S proteins. In some embodiments, the VLP includes the M, E, S, and N proteins.

In some embodiments, the VLP is an antigen presenting VLP in which a SARS-CoV-2 protein is attached to a protein scaffold. The antigen presenting VLP is produced as a fusion protein of the protein scaffold and a SARS-CoV-2 antigen as described herein. Suitable protein scaffolds for use in an antigen presenting VLP are known in the art. In some embodiments, the protein scaffold is Hepatitis B core antigen (HBc). In the antigen presenting VLP, the antigen may be bound to the major insertion region of HBc. In some embodiments, the antigen presenting VLP includes HBc and the SARS-CoV-2 S protein. In some embodiments, the antigen presenting VLP includes HBc and the SARS-CoV-2 S protein RBD. VLPs as described herein may be produced using any suitable method known in the art.

In some aspects, described herein are methods for producing a VLP in a mammalian cell. Mammalian cell-based systems for protein expression are known and described in the art. In general, a mammalian cell is transfected with a polynucleotide encoding a SARS-CoV-2 antigen or VLP and allowed to express said antigen or VLP from the polypeptide. The polynucleotides may be introduced into the mammalian cell using a construct or vector as described herein. Mammalian cells suitable for protein expression are known in the art, including, but not limited to, Chinese Hamster Ovary (CHO), Madin-Darby Canine Kidney (MDCK), Vero, and HEK 293T cells.

Vaccine compositions including the SARS-CoV-2 antigens, such as the VLPs described herein are also provided. As used herein “vaccine” refers to a composition that includes an antigen. Vaccine may also include a biological preparation that improves immunity to a particular disease. A vaccine may typically contain an agent, referred to as an antigen, that resembles a disease-causing microorganism, in this case SARS-CoV-2, and the agent may often be made from weakened or killed forms of the microbe, its toxins or one of its surface proteins. The antigen may stimulate the body's immune system to recognize the agent as foreign, destroy it, and “remember” it, so that the immune system can more easily recognize and destroy any of these microorganisms that it later encounters.

Vaccines may be prophylactic, e.g., to prevent or ameliorate the effects of a future infection by any natural or “wild” pathogen, or therapeutic, e.g., to treat the disease. Administration of the vaccine to a subject results in an immune response, generally against one or more specific diseases. The amount of a vaccine that is therapeutically effective may vary depending on the particular virus used, or the condition of the patient, and may be determined by a physician. The vaccine may be introduced directly into the subject by the subcutaneous, oral, oronasal, or intranasal routes of administration.

The vaccine compositions described herein also include a suitable carrier or vehicle for delivery. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

In another embodiment, the present formulation may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the SARS-CoV-2 antigens, or VLPs described herein.

The vaccine formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final vaccine formulation. The remainder of the vaccine formulation may be an acceptable diluent, to 100%, including water. The vaccine formulation may also be formulated as part of a water-in-oil, or oil-in-water emulsion.

In some embodiments, the present formulation may also comprise an adjuvant. An adjuvant is a substance or combination of substances that is used to increase the efficacy or potency of the formulation or modulates the immune response to a vaccine. An adjuvant may accelerate, prolong or enhance antigen-specific immune responses when used in combination with an antigen. An adjuvant may be an inorganic compound such as potassium alum, aluminum hydroxide, aluminum phosphate or calcium phosphate hydroxide. An adjuvant may also be an oil such as paraffin oil or propolis, a bacterial product such as killed Bordetella pertussis or Mycobacterium bovis or their toxoids, monophosphoryl lipid A or detoxified Salmonella minnesota lipopolysaccharide. An adjuvant may also be derived from plants, such as saponins from Quillaja (QS-21), soybean or Polygala senega. Cytokines such as IL-1, IL-2 or IL-12 may also act as adjuvants. Adjuvants may also include CpG DNA, Freund's complete or incomplete adjuvant or squalene including AS03 or MF59.

The vaccine formulation may be separated into vials or other suitable containers. The vaccine formulation herein described may then be packaged in individual or multi-dose ampoules or be subsequently lyophilized (freeze-dried) before packaging in individual or multi-dose ampoules. The vaccine formulation herein contemplated also includes the lyophilized version. The lyophilized vaccine formulation may be stored for extended periods of time without loss of viability at ambient temperatures. The lyophilized vaccine may be reconstituted by the end user and administered to a patient.

The term “lyophilization” or “lyophilized,” as used herein, refers to freezing of a material at low temperature followed by dehydration by sublimation, usually under a high vacuum. Lyophilization is also known as freeze drying. Many techniques of freezing are known in the art of lyophilization such as tray-freezing, shelf-freezing, spray-freezing, shell-freezing and liquid nitrogen immersion. Each technique will result in a different rate of freezing. Shell-freezing may be automated or manual. For example, flasks can be automatically rotated by motor driven rollers in a refrigerated bath containing alcohol, acetone, liquid nitrogen, or any other appropriate fluid. A thin coating of product is evenly frozen around the inside “shell” of a flask, permitting a greater volume of material to be safely processed during each freeze-drying run. Tray-freezing may be performed by, for example, placing the samples in lyophilizer, equilibrating 1 hr at a shelf temperature of 0° C., then cooling the shelves at 0.5° C./min to −40° C. Spray-freezing, for example, may be performed by spray-freezing into liquid, dropping by ˜20 μl droplets into liquid N₂, spray-freezing into vapor over liquid, or by other techniques known in the art.

Methods of inducing an immune response in a subject art also provided. A vaccine composition as described herein and including a SARS-CoV-2 antigen or VLP as described herein is administered to subject to induce an immune response. Following administration, the immune response of the subject may be tested using methods known in the art. To vaccine a subject, a therapeutically effective amount of a vaccine composition described herein is administered to the subject. The therapeutically effective amount of vaccine may typically be one or more doses, preferably in the range of about 0.01-10 mL, most preferably 0.1-1 mL, containing 1-500 micrograms, most preferably 1-100 micrograms of vaccine formulation/dose. The therapeutically effective amount may also depend on the vaccination species. For example, for smaller animals such as mice, a preferred dosage may be about 0.01-1 mL of a 1-50 microgram solution of antigen. For a human patient, a preferred dosage may be about 0.1-1 mL of a 1-50 microgram solution of antigen. The therapeutically effective amount may also depend on other conditions including characteristics of the patient (age, body weight, gender, health condition, etc.), and others.

The term “administration,” as used herein, refers to the introduction of a substance, such as a vaccine, into a subject's body. The administration, e.g., parenteral administration, may include subcutaneous administration, intramuscular administration, transcutaneous administration, intradermal administration, intraperitoneal administration, intraocular administration, intranasal administration, oral administration and intravenous administration.

The vaccine or the composition according to the invention may be administered to an individual according to methods known in the art. Such methods comprise application e.g. parenterally, such as through all routes of injection into or through the skin: e.g. intramuscular, intravenous, intraperitoneal, intradermal, mucosal, submucosal, or subcutaneous. Also, the vaccine may be applied by topical application as a drop, spray, gel or ointment to the mucosal epithelium of the eye, nose, mouth, anus, or vagina, or onto the epidermis of the outer skin at any part of the body.

Other possible routes of application are by spray, aerosol, or powder application through inhalation via the respiratory tract. In this last case, the particle size that is used will determine how deep the particles will penetrate into the respiratory tract.

Alternatively, application may be via the alimentary route, by combining with the food, feed or drinking water e.g. as a powder, a liquid, or tablet, or by administration directly into the mouth as a: liquid, a gel, a tablet, or a capsule, or to the anus as a suppository.

The present disclosure is generally applied to mammals, including but not limited to humans, cows, horses, sheep, pigs, goats, rabbits, dogs, cats, bats, mice and rats. In some embodiments, the present disclosure can be applied to birds. In certain embodiments, non-human mammals, such as mice and rats, may also be used for the purpose of demonstration. One may use the present invention for veterinary purposes. For example, one may wish to treat commercially important farm animals, such as cows, horses, pigs, rabbits, goats, sheep, and birds, such as chickens. One may also wish to treat companion animals, such as cats and dogs.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention.

### EXAMPLES

The following examples describe methods for making SARS-CoV-2 antigen virus-like particles (VLPs) in mammalian cells and use of said SARS-CoV-2 antigen VLPs.

### Example 1—Production of Virus-Like Particles

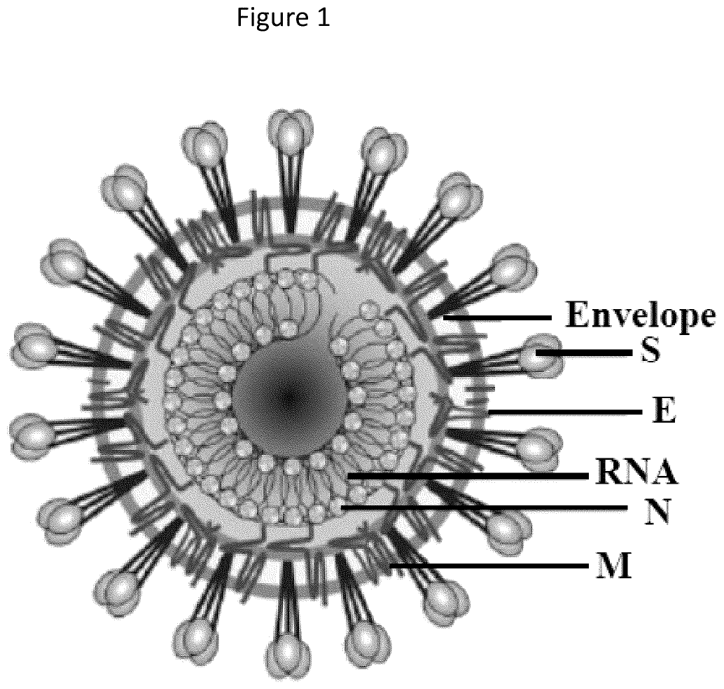

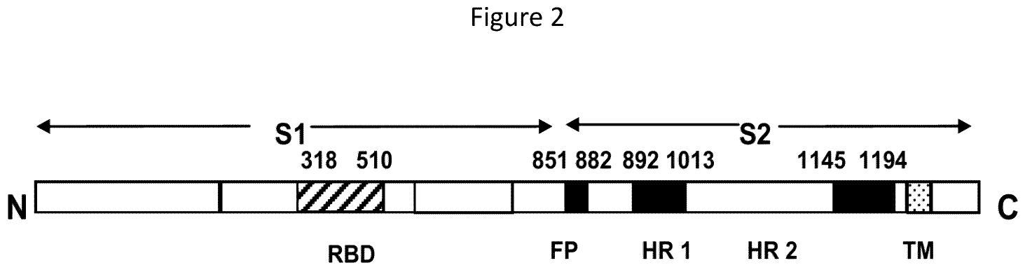

All CoVs have at least three envelope structural proteins, the membrane (M), spike (S), and envelope (E) (FIG. 1). The nucleocapsid (N) protein is an internal structural component that encapsulates the viral genome. The S protein is a glycoprotein that mediates receptor binding and fusion during entry into host cells [25]. Angiotensin-converting enzyme 2 (ACE2) is the receptor for SARS-CoV-2 and SARS-CoV [26-28]. S protein is the major target for neutralizing antibodies [29-32]. Many avian and mammalian coronavirus S protein are cleaved into S1 and S2 (FIG. 2). The receptor binding domain (RBD) on S proteins has been mapped for a number of CoVs, including SARS-CoV S [34, 35]. Sequence comparison of SARS-CoV and SARS-CoV-2 S proteins indicates approximately 76% identity for the full length S proteins, ˜73% identity for the RBD, but only ˜50% identity for the receptor binding motif core structure in the RBD [27]. This suggests that while both viruses use ACE2 for entry when infecting cells, receptor interactions may be structurally different, which could require specific targeting for effective vaccine development against SARS-CoV-2.

VLPs are an attractive platform for vaccine development. VLPs mimic virus particles, but lack the viral genome, and thus are a safe alternative for eliciting immune responses [36, 37]. Recombinant expression of the major structural proteins for many viruses results in VLP formation. Co-expression of only M and E is sufficient for VLP assembly, but S is incorporated when it is also expressed. In some cases, expression of the N gene enhances VLP production. Different protein combinations have been reported for production of SARS-CoV VLPs. VLPs are efficiently produced when M, E, and S are co-expressed in insect cells [38]. Co-expression of the M protein with N or E proteins was reported to be sufficient for VLP assembly in mammalian cells [39, 40], whereas another group showed that M must be expressed with N for efficient production and release of VLPs and that the S protein is incorporated when included [41]. Thus, the requirements for SARS-CoV VLP production are still not absolutely clear.

We hypothesize that delivery of the S protein or the receptor-binding domain (RBD) of the S protein on virus-like particles (VLPs) can stimulate production of protective levels of systemic and mucosal antibodies against SARS-CoV-2. The optimal requirement for VLP production in mammalian cells will be determined by transient expression of the membrane (M), envelope (E) and spike (S) proteins, with and without co-expression of the nucleocapsid (N) gene in 293T cells. Once requirements are confirmed VLP production will be scaled up by expression in MDCK and CHO cell suspension cultures. Purified VLPs will be characterized and used to evaluate systemic and mucosal immunogenicity in mice.

The optimal requirements for SARS-CoV-2 VLP production in mammalian cells will be determined as follows. SARS-CoV-2 M, E, S and N genes are being synthetically synthesized for expression in the pCAGGS vector under the control of the chicken beta-actin promoter, as we previously described [6-8]. Genes are codon optimized for expression in mammalian cells. The M gene will be co-expressed by transfection of 293T cells with the E and S genes, with and without the N gene to determine optimal requirements for efficient VLP assembly. Protein expression and extracellular VLPs will be characterized as previously described by SDS-PAGE and cryo-EM [6-8]. We have a panel of antibodies previously produced that recognize SARS- CoV E and M proteins and will be used since these will likely recognized regions of the protein that are conserved in the SARS-CoV-2 proteins. Once the optimal combination of genes is determined, we will assess VLP production in parallel in CHO-K1, Vero and 293T cells. These cells are used already to produce recombinant antibodies and VLP vaccines [43-49]. Supernatants will be clarified, concentrated and assessed for VLP concentration by SDS-PAGE and cryo-EM. The cells that yield the highest concentration will be used for small scale production of enveloped VLPs for immunogenicity measurement.

We have produced VLPs routinely as part of our assembly studies. We expect to quickly determine the optimal combination of genes that are required for VLP assembly. Small scale up is expected to provide sufficient VLPs for immunogenicity analysis in mice.

Although we routinely co-express genes in individual vectors for VLP production. It is possible that as we scale up VLP production that transfection of multiple vectors will not be efficient. As an alternative, genes will be cloned into available single vectors that allow for expression of 2-3 genes. The enveloped VLPs may not be sufficiently immunogenic without adjuvant. If this is the case, VLP immunization will be tested with acceptable adjuvants. If current antibodies in the lab do not cross react with SARS-CoV-2 proteins, we will generate antibodies. Alternatively, we expect that commercial antibodies will become available.

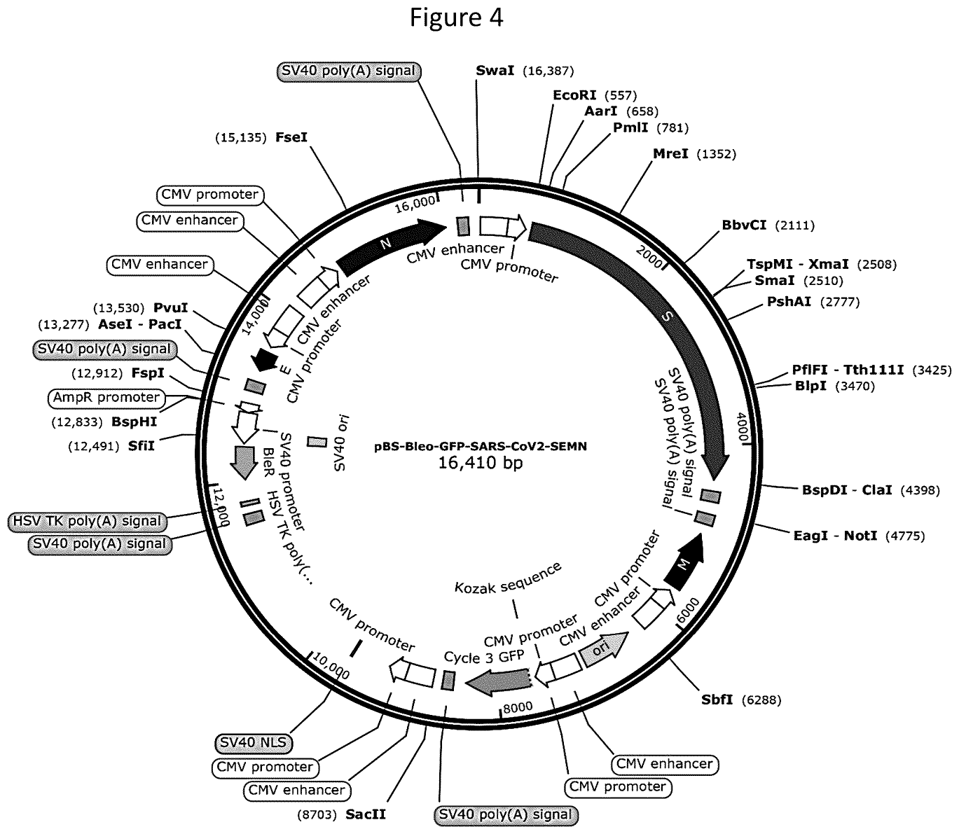

Additionally, a second-generation expression system for SARS-2 VLP production is being constructed. The new vector expresses the M, E, S and N genes together from individual promoters (see vector map in FIG. 4). Transfection of one vector assures that all cells will express all four SARS-2 genes. Additionally, the vector contains a bleomycin resistance gene, which will allow for stable expression of the genes and should result in higher VLP production. FDA approved CHO cells will be used for expression.

### Example 2—Expression of Virus-Like Particles

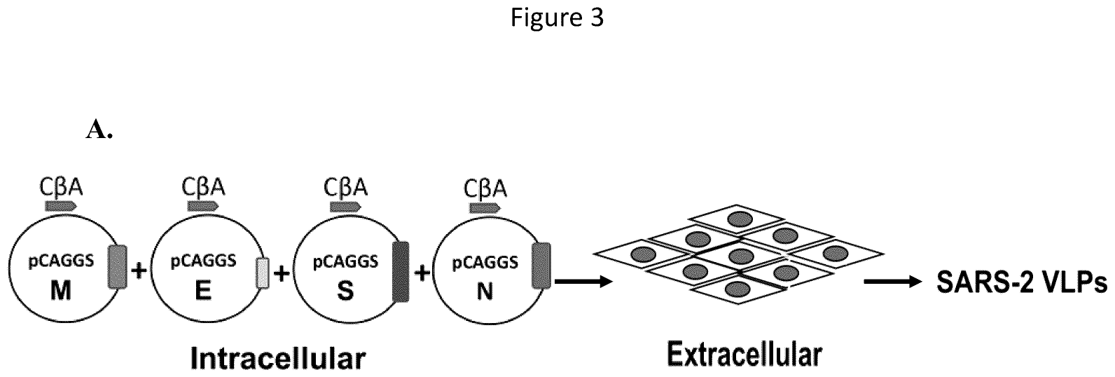

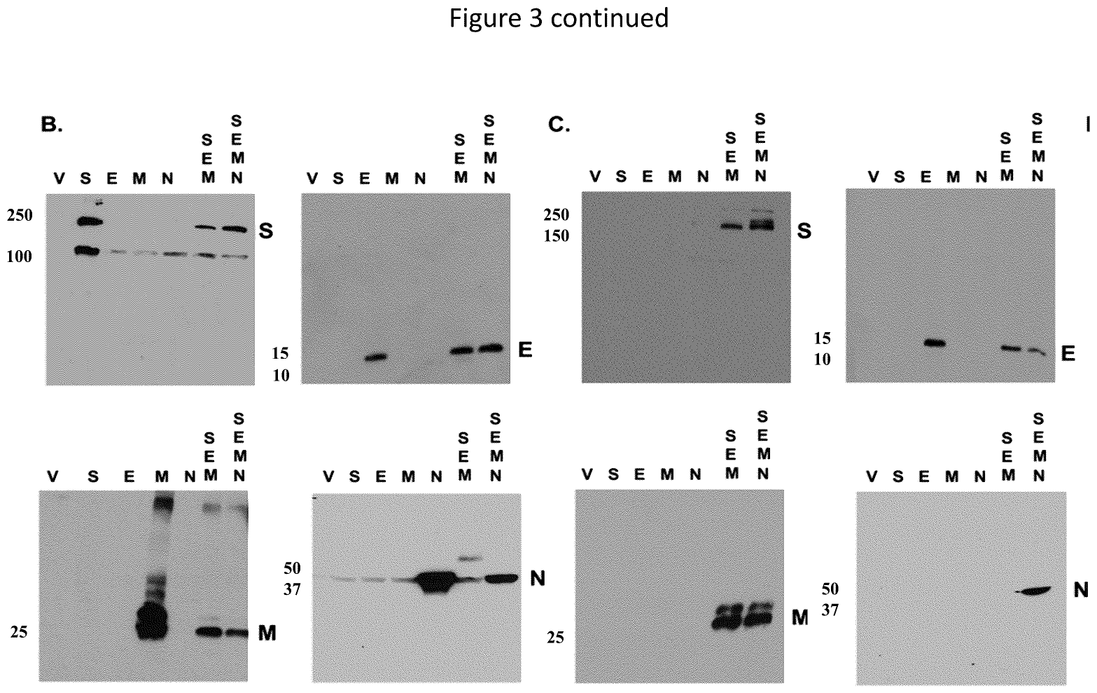

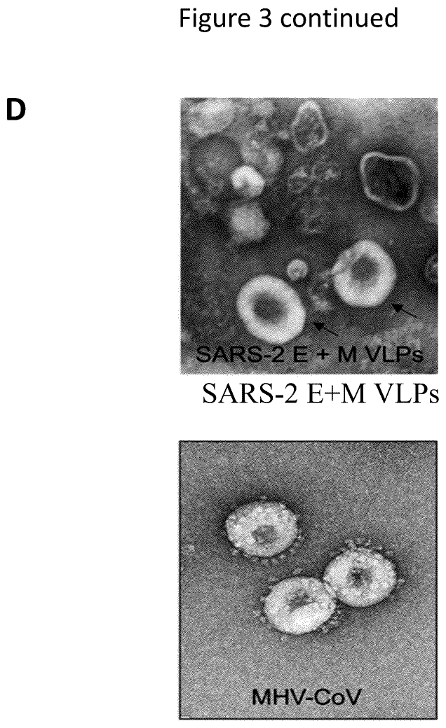

We have shown that SARS-CoV-2 virus-like-particles (VLPs) assemble when the viral envelope (E, M, S) and nucleocapsid (N) proteins are co-expressed singly from individual plasmids (FIG. 3). These data support the use of VLPs as a platform for some of our proposed studies. Furthermore, the presence of E in the extracellular fraction, when E is expressed alone, is consistent with our hypothesis that this protein promotes membrane curvature, allowing for release from cells (likely as E vesicles).

### Example 3—Evaluation of Immunogenicity

To evaluate immunogenecitiy of VLPs, we will determine neutralizing activity of immunized sera. Mice will be immunized systemically and mucosally VLPs and serum and mucosal washes will be analyzed for induction of binding and neutralizing antibodies to 2019-nCoV. Immunogens which induce neutralizing antibodies will be optimized by addition of adjuvants and/or prime-boost strategies to determine optimal formulations/regimens.

Systemic immunization. Balb/c mice will be immunized systemically (i.m.) in the absence or presence of alum adjuvant with selected VLPs. Alum will be used with the VLPs because it has been approved for use as an adjuvant in injectable human vaccines (such as recombinant HBsAg vaccines) and can induce a strong Th2-biased antibody response. Immunity induced by replicating pox vectors is usually not enhanced by the presence of an adjuvant (unpublished observations). Serum samples will be collected and analyzed for the presence of spike and RBD specific serum IgG by antigen-specific ELISAs. Serum IgG1 and IgG2a will also be analyzed to determine the type (Th1 vs Th2) of the immune response to individual immunogen/adjuvant combinations. Finally, serum samples will be assayed for neutralizing capacity using VSV pseudotyped with SARS-Coronavirus-2 S protein.

Mucosal immunization. Balb/c mice will be immunized intranasally in the absence or presence of synthetic CpG oligodeoxynucleotides (ODN) with selected VLPs.

If weak mucosal immune responses are observed following intranasal immunization only, we will try an intranasal priming/systemic boosting strategy which has been shown to be effective in augmenting sIgA production [89].

Neutralization assays. SARS-Coronavirus-2 neutralizing capacity of the serum and mucosal antibodies generated will be analyzed using VSV pseudotyped with SARS-Coronavirus-2 S protein.

If weak mucosal responses are observed for our VLP candidates, we will try other mucosal adjuvants (including LT mutant (R192G), saponin [90], and IVX-908 proteosome formulation (Protollin) [91, 92]. It is possible that antibodies against the RBD will not be sufficient to achieve the desired level of protection. If this is the case other neutralizing epitopes that have been identified in S will be incorporated into the VLPs and used in conjunction with the RBD VLPs or as part of a prime-boost regimen.

### Example 4—Establishment of an Adapted Mouse Model for VLP Vaccine Studies

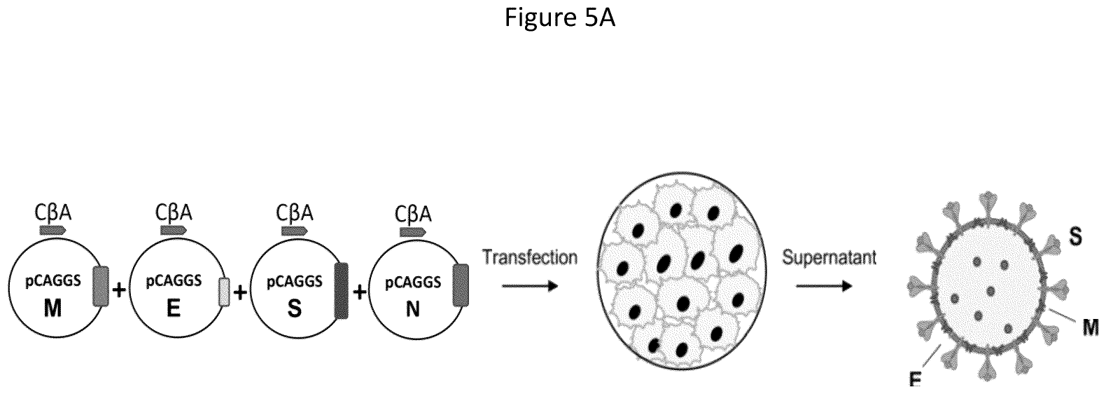

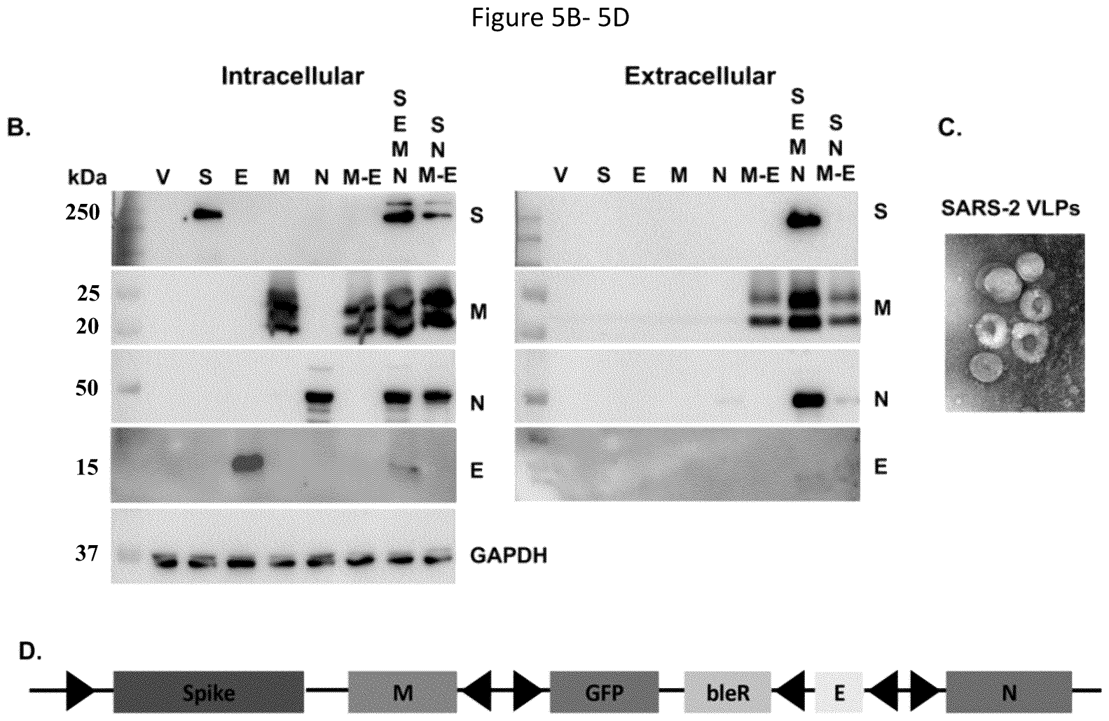

SARS-2 structural genes optimized for expression in mammalian cells were synthesized and cloned into a pCAGGS vector under the control of the chicken beta-actin promoter (FIG. 5). We initially used the Wuhan SARS-2 S protein for determining the requirements for VLP assembly, but subsequently modified the S clone to remove the furin cleavage site and also introduce two proline substitutions in the S2 subunit to stabilize S in the prefusion state. The S gene was further modified to include six proline (HexaPro) substitutions to further stabilize the protein (42, 95, 96) (SEQ ID NO: 14). Co-expression of the genes is driven by transfection of the individual plasmids into 293T cells. The —100 nm diameter SARS-2 VLPs produced in 293T cells are morphologically and biochemically like SARS-CoV-2 virus particles based on TEM, NanoSight measurements, protein profiles and posttranslational modifications (FIG. 5). Western blotting confirmed VLP formation showing S, M and N proteins are not released into the extracellular media when expressed singly, but M is present when co-expressed with E (FIG. 5).

In addition to the single plasmids, we have constructed a plasmid that contains all four (M, E, N, S) genes to facilitate assurance of co-expression of all genes in single cells. One of the plasmid constructs is shown as an example in FIG. 5D. Different combinations of three of the four genes have also been constructed with and without GFP and bleomycin (bleR) for monitoring transfection efficiency and selection of stable expression for VLP production. VLP are produced from these constructs, but with a lower yield as compared to single plasmids. This effect can be seen in FIG. 5B where M and E are co-expressed.

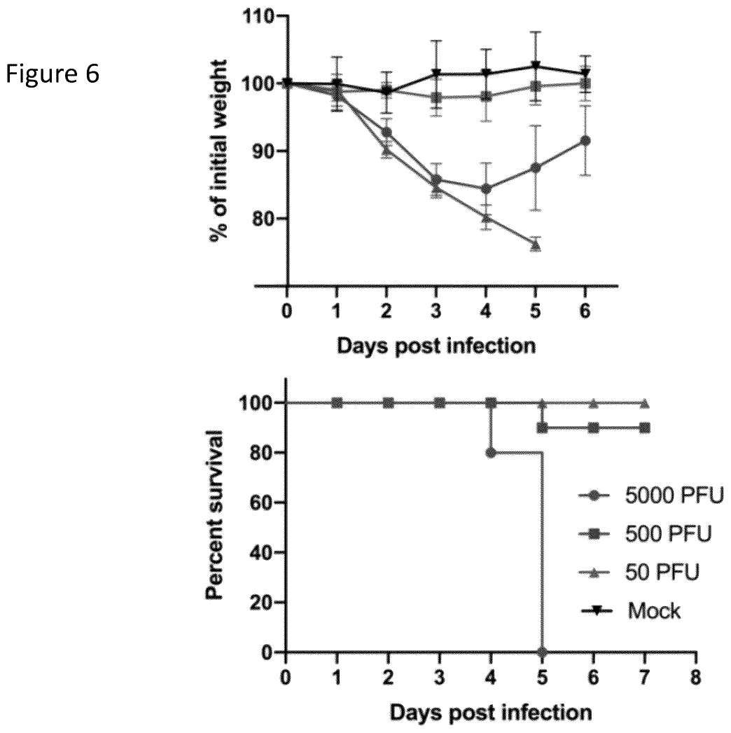

Next, we tested the immunogenicity of the VLP. A virulent mouse-adapted SARS2-N501YMA30 model was created by adopting SARS-2 mice with introduction of N501Y substitution in the S gene and 30 subsequent passages in BALB/c mice (93). We obtained the MA30 virus and established the model last year in the Biodesign Center for Immunotherapy, Vaccines and Virotherapy (CIVV). The model was used for initial testing of a vaccinia vaccine platform (94). The model has also now been extended to infection of C57/B16 mice for use in this study. FIG. 6 shows weight loss and survival in C57/B16 mice inoculated intranasally with MA30.

### REFERENCES

25. Li, F., Structure, Function, and Evolution of Coronavirus Spike Proteins. Annu Rev Virol, 2016. 3(1): p. 237-261.
26. Lu, R., et al., Genomic characterisation and epidemiology of 2019 novel coronavirus: implications for virus origins and receptor binding. Lancet, 2020.
27. Wan, Y., et al., Receptor recognition by novel coronavirus from Wuhan: An analysis based on decade-long structural studies of SARS. J Virol, 2020.
28. Zhou, P., et al., A pneumonia outbreak associated with a new coronavirus of probable bat origin. Nature, 2020.
29. Jiang, L., et al., Potent neutralization of MERS-CoV by human neutralizing monoclonal antibodies to the viral spike glycoprotein. Sci Transl Med, 2014. 6(234): p. 234ra59.
30. Ying, T., et al., Exceptionally potent neutralization of Middle East respiratory syndrome coronavirus by human monoclonal antibodies. J Virol, 2014. 88(14): p. 7796-805.
31. Tai, W., et al., Recombinant Receptor-Binding Domains of Multiple Middle East Respiratory Syndrome Coronaviruses (MERS-CoVs) Induce Cross-Neutralizing Antibodies against Divergent Human and Camel MERS-CoVs and Antibody Escape Mutants. J Virol, 2017. 91(1).
32. Niu, P., et al., Ultrapotent Human Neutralizing Antibody Repertoires Against Middle East Respiratory Syndrome Coronavirus From a Recovered Patient. J Infect Dis, 2018. 218(8): p. 1249-1260.
33. Xiao, X., et al., The SARS-CoV S glycoprotein: expression and functional characterization. Biochem.Biophys.Res.Commun., 2003. 312(4): p. 1159-1164.
34. Li, W., et al., Angiotensin-converting enzyme 2 is a functional receptor for the SARS coronavirus. Nature, 2003. 426(6965): p. 450-454.
35. Li, F., Receptor recognition mechanisms of coronaviruses: a decade of structural studies. J Virol, 2015. 89(4): p. 1954-64.
36. Lopez-Macias, C., et al., Safety and immunogenicity of a virus-like particle pandemic influenza A (H1N1) 2009 vaccine in a blinded, randomized, placebo-controlled trial of adults in Mexico. Vaccine, 2011. 29(44): p. 7826-34.
37. Mohsen, M.O., et al., Major findings and recent advances in virus-like particle (VLP)-based vaccines. Semin Immunol, 2017. 34: p. 123-132.
38. Mortola, E. and P. Roy, Efficient assembly and release of SARS coronavirus-like particles by a heterologous expression system. FEBS Lett., 2004. 576(1-2): p. 174-178.
39. Tseng, Y. T., et al., Self-assembly of severe acute respiratory syndrome coronavirus membrane protein. J.Biol.Chem., 2010. 285(17): p. 12862-12872.
40. Huang, Y., et al., Generation of synthetic severe acute respiratory syndrome coronavirus pseudoparticles: implications for assembly and vaccine production. J.Virol., 2004. 78(22): p. 12557-12565.
41. Siu, Y.L., et al., The M, E, and N structural proteins of the severe acute respiratory syndrome coronavirus are required for efficient assembly, trafficking, and release of virus-like particles. J.Virol., 2008. 82(22): p. 11318-11330.
42. Niwa, H., K. Yamamura, and J. Miyazaki, Efficient Selection for High-Expression Transfectants with A Novel Eukaryotic Vector. Gene, 1991. 108(2): p. 193-199.
43. Dhara, V.G., et al., Recombinant Antibody Production in CHO and NS0 Cells: Differences and Similarities. BioDrugs, 2018. 32(6): p. 571-584.
44. Hofmann, I., et al., Expression of the human cytomegalovirus pentamer complex for vaccine use in a CHO system. Biotechnol Bioeng, 2015. 112(12): p. 2505-15.
45. Montomoli, E., et al., Cell culture-derived influenza vaccines from Vero cells: a new horizon for vaccine production. Expert Rev Vaccines, 2012. 11(5): p. 587-94.
46. Barrett, P.N., D. Portsmouth, and H.J. Ehrlich, Vero cell culture-derived pandemic influenza vaccines: preclinical and clinical development. Expert Rev Vaccines, 2013. 12(4): p. 395-413
47. Garg, H., et al., Development of Virus-Like-Particle Vaccine and Reporter Assay for Zika Virus. J Virol, 2017. 91(20).
48. Cervera, L., et al., Generation of HIV-1 Gag VLPs by transient transfection of HEK 293 suspension cell cultures using an optimized animal-derived component free medium. J Biotechnol, 2013. 166(4): p. 152-65.
49. Buffin, S., et al., Influenza A and B virus-like particles produced in mammalian cells are highly immunogenic and induce functional antibodies. Vaccine, 2019. 37(46): p. 6857-6867.
89. Matoba, N., et al., Humoral immune responses by prime-boost heterologous route immunizations with CTB-MPR(649-684), a mucosal subunit HIV/AIDS vaccine candidate. Vaccine, 2006. 24(23): p. 5047-55.
90. Pickering, R.J., et al., Crude saponins improve the immune response to an oral plant-made measles vaccine. Vaccine, 2006. 24(2): p. 144-50.
91. Fries, L.F., et al., Safety and immunogenicity of a proteosome-Shigella flexneri 2a lipopolysaccharide vaccine administered intranasally to healthy adults. Infect Immun, 2001. 69(7): p. 4545-53.
92. Orr, N., et al., Enhancement of anti-Shigella lipopolysaccharide (LPS) response by addition of the cholera toxin B subunit to oral and intranasal proteosome-Shigella flexneri 2a LPS vaccines. Infect Immun, 1994. 62(11): p. 5198-200.
93. Roy Wong LY, et al. Eicosanoid signaling as a therapeutic target in middle-aged mice with severe COVID-19 . bioRxiv. 2021.
94. Kibler KV. et al. Replication-Competent NYVAC-KC Yields Improved Immunogenicity to HIV-1 Antigens in Rhesus Macaques Compared to Nonreplicating NYVAC. J Virol. 2019;93(3).
95. Wrapp D. et al., EM structure of the 2019-nCoV spike in the prefusion conformation. Science. 2020; 367 (6483): 1260-3.
96. Chang CK. et al., The dimer interface of the SARS coronavirus nubleocapsid protein adapts a porcine respiratory and reproductive syndrome virus-like structure. FEBS Lett. 2005; 579(25):5663-8.

### Embodiments of the Invention

Embodiment 1: A Severe Acute Respiratory Syndrome Coronavirus 2 (SARS-CoV-2) virus-like particle (VLP) comprising SEQ ID NO:7 and SEQ ID NO:8 and sequences at least 90% identical thereto, and combinations thereof, and lacking SARS-CoV-2 viral genome and optionally further comprising SEQ ID NO: 1 or sequences 90% identical thereto.

Embodiment 2: The SARS-CoV-2 VLP of embodiment 1, additionally comprising SEQ ID NO:9or a sequence at least 90% identical thereto.

Embodiment 3: The SARS-CoV-2 VLP of embodiment 1 or 2, wherein the sequence is selected from the group consisting of a mutant SARS-CoV-2 Spike or RBD of SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38 and sequences at least 90% identical thereto, and combinations thereof.

Embodiment 4: A method for producing the SARS-CoV-2 VLP of embodiment 1-3 comprising transfecting a mammalian cell with (i) a first polynucleotide encoding SEQ ID NO:1 or a sequence at least 90% identical thereto; (ii) a second polynucleotide encoding SEQ ID NO:7 or a sequence at least 90% identical thereto; and (iii) a third polynucleotide encoding SEQ ID NO:8 or a sequence at least 90% identical thereto; and extracting the SARS-CoV-2 VLP from the mammalian cell.

Embodiment 5: The method of embodiment 3, additionally comprising transfecting the mammalian cell with (iv) a fourth polynucleotide encoding SEQ ID NO:9 or a sequence at least 95% identical thereto.

Embodiment 6: The method of embodiment 3 or 4, wherein the sequence is selected from the group consisting of a mutant SARS-CoV-2 Spike or RBD of SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38 and sequences at least 90% identical thereto, and combinations thereof.

Embodiment 7: The method of embodiment 4 or 5, wherein the first, second, third, and/or fourth polynucleotide is codon optimized for expression in a mammalian cell.

Embodiment 8: The method of any of embodiments 4-7, wherein the first, second, and third polynucleotide are transfected into the mammalian cell using at least one vector.

Embodiment 9: The method of any of embodiments 4-8, wherein a single vector comprises the first, second, and third polynucleotides.

Embodiment 10: The method of any embodiments 4-9, wherein the mammalian cell is transfected with a first vector comprising the first polynucleotide, a second vector comprising the second polynucleotide, and a third vector comprising the third polynucleotide.

Embodiment 11: The method of any of embodiments 8-10, wherein the vector additionally comprises a promoter.

Embodiment 12: The method of embodiment 11, wherein the promoter is a chicken β-actin promoter or the CMV promoter.

Embodiment 13: The method of any of embodiment 8-12, wherein the vector additionally comprises a selection marker.

Embodiment 14: The method of any of embodiments 4-13, wherein the mammalian cell is selected from the group consisting of Chinese Hamster Ovary (CHO), Madin-Darby Canine Kidney (MDCK), Vero and HEK 293T.

Embodiment 15: A vaccine composition comprising the SARS-CoV-2 VLP of any of embodiments 1-3 and a pharmaceutically acceptable carrier.

Embodiment 16: The vaccine composition of embodiment 15, additionally comprising an adjuvant.

Embodiment 17: The vaccine composition of embodiment 16, wherein the adjuvant is a mucosal adjuvant selected from the group consisting of LT mutant R1925G, saponin, and IVX-908 proteosome formulation.

Embodiment 18: A method for inducing an immune response in a host comprising administering an effective amount of the vaccine composition of any of embodiments 15-17 to the host.

Embodiment 19: The method of embodiment 18, wherein the host is human.

Embodiment 20: The method of embodiment 18 or 19, wherein the vaccine composition is administered systemically.

Embodiment 21: The method of embodiment 18 or 19, wherein the vaccine composition is administered by injection.

Embodiment 22: The method of embodiment 18 or 19, wherein the vaccine composition is administered intranasally.

Embodiment 23: The method of embodiment 18 or 19, wherein the vaccine composition additionally comprises an adjuvant.

1. A severe acute respiratory syndrome coronavirus 2 (SARS-CoV-2) virus-like particle (VLP) comprising SEQ ID NO:7 and SEQ ID NO:8 or sequences at least 90% identical to SEQ ID NO: 7 and SEQ ID NO: 8, and combinations thereof, and lacking SARS-CoV-2 viral genome and optionally further comprising SEQ ID NO: 1 or sequences 90% identical to SEQ ID NO: 1.

2. The SARS-CoV-2 VLP of claim 1, additionally comprising SEQ ID NO:9 or a sequence at least 90% identical thereto.

3. The SARS-CoV-2 VLP of claim 1, wherein the sequence is selected from the group consisting of a mutant SARS-CoV-2 Spike or RBD of SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38 and sequences at least 90% identical thereto, and combinations thereof

4. A method for producing the SARS-CoV-2 VLP of claim 1 comprising
transfecting a mammalian cell with (i) a first polynucleotide encoding SARS-CoV-2 S protein (SEQ ID NO:1) or a sequence at least 90% identical thereto; (ii) a second polynucleotide encoding SARS-CoV-2 M protein (SEQ ID NO:7) or a sequence at least 90% identical thereto; and (iii) a third polynucleotide encoding SARS-CoV-2 E protein (SEQ ID NO:8) or a sequence at least 90% identical thereto; and
extracting the SARS-CoV-2 VLP from the mammalian cell.

5. The method of claim 4, additionally comprising transfecting the mammalian cell with (iv) a fourth polynucleotide encoding SARS-CoV-2 N protein (SEQ ID NO:9) or a sequence at least 90% identical thereto.

6. The method of claim 4, wherein the sequence is selected from the group consisting of a mutant SARS-CoV-2 Spike or RBD of SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38 and sequences at least 90% identical thereto, and combinations thereof.

7. The method of claim 4, wherein the first, second, and third polynucleotide are codon optimized for expression in a mammalian cell.

8. The method of claim 4, wherein the first, second, and third polynucleotide are transfected into the mammalian cell using at least one vector.

9. The method of claim 8, wherein a single vector comprises the first, second, and third polynucleotides, or wherein the mammalian cell is transfected with a first vector comprising the first polynucleotide, a second vector comprising the second polynucleotide, and a third vector comprising the third polynucleotide.

10. (canceled)

11. The method of claim 8, wherein the vector additionally comprises a promoter.

12. The method of claim 11, wherein the promoter is a chicken β-actin promoter or the CMV promoter.

13. The method of claim 8, wherein the vector additionally comprises a selection marker.

14. The method of claim 4, wherein the mammalian cell is selected from the group consisting of Chinese Hamster Ovary (CHO), Madin-Darby Canine Kidney (MDCK), Vero and HEK 293T.

15. A vaccine composition comprising the SARS-CoV-2 VLP of claim 1 and a pharmaceutically acceptable carrier.

16. The vaccine composition of claim 15, additionally comprising an adjuvant.

17. The vaccine composition of claim 16, wherein the adjuvant is a mucosal adjuvant selected from the group consisting of LT mutant R1925G, saponin, and IVX-908 proteosome formulation.

18. A method for inducing an immune response in a host comprising administering an effective amount of the vaccine composition of claim 15 to the host.

19. The method of claim 18, wherein the host is human.

20. (canceled)

21. The method of claim 18, wherein the vaccine composition is administered intranasally or by injection.

22. (canceled)

23. The method of claim 18, wherein the vaccine composition additionally comprises an adjuvant.


SEQUENCE LISTING (38 sequences)

SEQ ID NO: 1 - protein, 1273 aa, Severe Acute Respiratory Syndrome Coronaviruses 2
  MISC_FEATURE (1)..(1273): Spike
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 150 aa, Artificial Sequence
  Synthetic- C7L polypeptide
MGIQHEFDIIINGDIALRNLQLHKGDNYGCKLKIISNDYKKLKFRFIIRPDWSEIDEVKGLTVFANNYAVKVNKVDDTFYYVIYEAVIHLYNKKTEILIYSDDENELFKHYYPYISLNMISKKYKVKEENYSSPYIEHPLIPYRDYESMD

SEQ ID NO: 3 - protein, 284 aa, Artificial Sequence
  Synthetic- K1L polypeptide
MDLSRINTWKSKQLKSFLSSKDTFKADVHGHSALYYAIADNNVRLVCTLLNAGALKNLLENEFPLHQAATLEDTKIVKILLFSGMDDSQFDDKGNTALYYAVDSGNMQTVKLFVKKNWRLMFYGKTGWKTSFYHAVMLNDVSIVSYFLSEIPSTFDLAILLSCIHTTIKNGHVDMMILLLDYMTSTNTNNSLLFIPDIKLAIDNKDIEMLQALFKYDINIYSVNLENVLLDDAEITKMIIEKHVEYKSDSYTKDLDIVKNNKLDEIISKNKELRLMYVNCVKKN

SEQ ID NO: 4 - DNA, 11 bp, Artificial Sequence
  Synthetic- TEE
aaaactgctaa

SEQ ID NO: 5 - DNA, 26 bp, Artificial Sequence
  Synthetic- viral synthetic late promoter
catattgaagagacagagtgatatat

SEQ ID NO: 6 - protein, 193 aa, Artificial Sequence
  Synthetic- RBD
FRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 7 - protein, 222 aa, Severe Acute Respiratory Syndrome Coronaviruses 2
  MISC_FEATURE (1)..(222): M protein of SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 75 aa, Severe Acute Respiratory Syndrome Coronaviruses 2
  MISC_FEATURE (1)..(75): E protein of SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 9 - protein, 419 aa, Severe Acute Respiratory Syndrome Coronaviruses 2
  MISC_FEATURE (1)..(419): N protein of SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 10 - DNA, 37 bp, Artificial Sequence
  Synthetic- TEE
catattgaagagacagagtgatatataaaactgctaa

SEQ ID NO: 11 - DNA, 42 bp, Artificial Sequence
  Synthetic- TEE
agaaccatattgaagagacagagtgatatataaaactgctaa

SEQ ID NO: 12 - DNA, 90 bp, Artificial Sequence
  Synthetic- TEE
agaaccatattgaagagacagagtgatatataaaactgctaactcaagcagcacaagaattaaatgaataccaagaaaatacttggccag

SEQ ID NO: 13 - protein, 9 aa, Artificial Sequence
  Synthetic- ebola glycoprotein 6D8 epitope tag
VYKLDISEA

SEQ ID NO: 14 - DNA, 3846 bp, Artificial Sequence
  Synthetic- Modified spike of SARS-CoV-2 with furin cleavage site and stabilizing proline mutations
gaattcaggaggtccaccatgttcgtgttcctggtgcttctgcctctggttagcagccagtgcgtgaacctgaccacccggacccagctgccacctgcttacacaaacagcttcaccagaggcgtgtactaccccgataaggtgttcagaagcagcgtgctgcacagcacacaggacctgttcctgcctttcttcagcaacgtgacatggttccacgccatccacgtgtccggcacaaacggcacaaagcggttcgataaccctgtgctacctttcaatgacggcgtgtattttgcctctaccgagaagtctaacatcattcggggctggatcttcggcaccaccctggacagcaaaacccagagcctgctgatcgtgaacaacgccactaacgtggtgatcaaagtttgtgaatttcagttctgcaacgacccgtttctgggcgtttactaccacaagaacaacaagagctggatggaatccgagttcagagtgtactcgtctgccaacaactgtaccttcgagtacgtgtcccagcccttcctgatggacctggaaggcaagcagggcaacttcaagaatctgagagagtttgtgttcaagaatatcgacggctacttcaagatctacagcaagcacaccccgatcaacctggtgcgagatctgccccaaggcttcagcgccctggaaccactggttgacctgcccatcggaatcaacattacgcggttccaaacgctgctggccctgcatagaagctacctgacacctggagattccagctccggctggaccgccggcgccgctgcctactacgtgggctacctgcagcctagaaccttcctgctgaaatataatgagaacggcaccatcacagacgccgtggactgtgccctggatcccttgtctgagacaaagtgcacactgaagagcttcacagtggaaaaaggcatctaccaaacatccaacttccgggtccagcctacagaaagcatcgtgcggtttcccaacatcaccaatctgtgtccttttggcgaggttttcaatgcaacaagattcgcttctgtctacgcctggaatcggaagagaatctctaactgcgtggccgattacagcgttctgtacaacagcgccagcttttccacctttaagtgctacggcgtcagtcctaccaagctgaacgacttgtgcttcaccaacgtgtacgccgactccttcgtgatccggggcgacgaggtgcggcagatcgcccctgggcagaccggcaagatcgcggattacaactacaagctgcctgatgattttaccggatgcgtgattgcttggaacagcaacaacctggactctaaggttggcggaaactacaactacctgtatagactgtttagaaagtccaacctgaagcccttcgaaagagacatcagcacagagatctaccaggccggcagtaccccatgtaatggtgttgagggcttcaactgctacttccctctgcagtcttacggcttccagcctacaaacggcgtgggctaccagccctacagagtggtggtcctcagcttcgagctcctgcacgctcctgccaccgtctgcggtcctaagaagagcacaaacctggtgaagaataagtgcgtgaatttcaactttaacggcctgaccggcaccggagtgctgacggaaagcaacaaaaagttcctgcccttccagcaatttggaagagatatcgctgacaccacagacgctgtgcgggatccgcagactctggaaatcctggacatcaccccctgttctttcggaggcgtgagcgtgatcacgcctggcaccaacaccagcaaccaggtggccgtgctgtaccaggacgttaattgcaccgaggtgccagttgccatccacgccgatcagctgaccccaacatggcgggtgtattctacaggtagcaacgtgttccagacccgggccggctgcctgatcggcgccgagcacgtcaataatagctacgagtgcgacatccccattggagctggcatctgcgccagctaccagacgcaaacaaacagccctggcagcgccagcagcgtggccagtcagagtatcatcgcttacaccatgagcctgggcgccgaaaacagcgtggcctattctaacaatagcatcgccatccctaccaacttcaccatctctgtgacaaccgagattctgcctgtatccatgacaaagaccagtgtcgactgcactatgtacatctgcggcgatagcaccgaatgttctaacctcctgttgcagtatggaagcttttgtactcagctcaacagagccctgacaggcattgctgtggaacaggataagaacacccaggaggtgttcgcccaagtgaagcagatctataagacacctcctatcaaggacttcggcggcttcaactttagccagatcctgcctgacccgagcaagcctagcaaaaggtcccccatcgaggaccttctcttcaacaaggtgaccttggccgacgccggatttatcaagcagtacggcgactgcctgggagatatcgccgctagagatctgatttgtgctcaaaagttcaatggcctgaccgtgttaccgcctctgttgaccgatgagatgatcgcccagtacaccagcgccctgctggccggaacaatcaccagcggctggacattcggtgccggccccgccctgcaaatccccttccccatgcagatggcctaccggttcaatggtatcggcgtgacacaaaacgtgctgtacgagaaccagaaactgatcgccaaccagttcaacagcgccatcggaaagatccaggattctctgtcttctacccccagtgccctgggcaagctgcaagacgtggtcaaccagaacgcccaggccctgaacacactggtgaaacagctgagctccaacttcggcgccatcagctccgtactgaatgacatcctgtcaagactggatccccccgaagccgaggtgcagattgatagactgatcaccggcagactgcagagcctgcagacatacgtgacccagcagctgatccgggctgctgagatcagagctagtgctaatctcgccgctacaaagatgagcgaatgcgtgctgggacagagcaagagagtggacttctgcggcaaggggtatcacctgatgtccttcccacagagcgcacctcacggcgtggtgtttttacatgtgacctacgtgcctgcccaggagaagaactttaccacggcccctgctatctgccacgatggcaaagcccacttccccagagagggcgtgttcgtaagcaacggcacacactggtttgtgacccagagaaacttctacgagccccagatcatcaccaccgacaacaccttcgtgtctggcaattgtgatgtggtgatcggcatcgtgaacaacaccgtgtacgaccctctgcagcccgagctggacagcttcaaagaggaactggacaagtacttcaagaaccacaccagccctgatgtggacctcggagacatcagcggcatcaacgcctccgtggtgaacatccagaaggaaattgacagactgaatgaggtggccaagaaccttaatgagagcctgatcgacctccaggagctgggcaagtacgagcagtacatcaagtggccctggtacatctggctggggttcattgcaggactgatcgccatcgtgatggtgaccatcatgctgtgctgcatgacctcttgttgtagctgcctgaagggctgctgctcctgcggcagctgttgcaagttcgacgaggatgacagcgagcctgtgctgaaaggcgtgaagctgcactacacctgaatcgat

SEQ ID NO: 15 - DNA, 3846 bp, Artificial Sequence
  Synthetic- Condon optimized Spike of SARS-Co-V-2
gaattcaggaggtccaccatgttcgtgttcctggtgcttctgcctctggttagcagccagtgcgtgaacctgaccacccggacccagctgccacctgcttacacaaacagcttcaccagaggcgtgtactaccccgataaggtgttcagaagcagcgtgctgcacagcacacaggacctgttcctgcctttcttcagcaacgtgacatggttccacgccatccacgtgtccggcacaaacggcacaaagcggttcgataaccctgtgctacctttcaatgacggcgtgtattttgcctctaccgagaagtctaacatcattcggggctggatcttcggcaccaccctggacagcaaaacccagagcctgctgatcgtgaacaacgccactaacgtggtgatcaaagtttgtgaatttcagttctgcaacgacccgtttctgggcgtttactaccacaagaacaacaagagctggatggaatccgagttcagagtgtactcgtctgccaacaactgtaccttcgagtacgtgtcccagcccttcctgatggacctggaaggcaagcagggcaacttcaagaatctgagagagtttgtgttcaagaatatcgacggctacttcaagatctacagcaagcacaccccgatcaacctggtgcgagatctgccccaaggcttcagcgccctggaaccactggttgacctgcccatcggaatcaacattacgcggttccaaacgctgctggccctgcatagaagctacctgacacctggagattccagctccggctggaccgccggcgccgctgcctactacgtgggctacctgcagcctagaaccttcctgctgaaatataatgagaacggcaccatcacagacgccgtggactgtgccctggatcccttgtctgagacaaagtgcacactgaagagcttcacagtggaaaaaggcatctaccaaacatccaacttccgggtccagcctacagaaagcatcgtgcggtttcccaacatcaccaatctgtgtccttttggcgaggttttcaatgcaacaagattcgcttctgtctacgcctggaatcggaagagaatctctaactgcgtggccgattacagcgttctgtacaacagcgccagcttttccacctttaagtgctacggcgtcagtcctaccaagctgaacgacttgtgcttcaccaacgtgtacgccgactccttcgtgatccggggcgacgaggtgcggcagatcgcccctgggcagaccggcaagatcgcggattacaactacaagctgcctgatgattttaccggatgcgtgattgcttggaacagcaacaacctggactctaaggttggcggaaactacaactacctgtatagactgtttagaaagtccaacctgaagcccttcgaaagagacatcagcacagagatctaccaggccggcagtaccccatgtaatggtgttgagggcttcaactgctacttccctctgcagtcttacggcttccagcctacaaacggcgtgggctaccagccctacagagtggtggtcctcagcttcgagctcctgcacgctcctgccaccgtctgcggtcctaagaagagcacaaacctggtgaagaataagtgcgtgaatttcaactttaacggcctgaccggcaccggagtgctgacggaaagcaacaaaaagttcctgcccttccagcaatttggaagagatatcgctgacaccacagacgctgtgcgggatccgcagactctggaaatcctggacatcaccccctgttctttcggaggcgtgagcgtgatcacgcctggcaccaacaccagcaaccaggtggccgtgctgtaccaggacgttaattgcaccgaggtgccagttgccatccacgccgatcagctgaccccaacatggcgggtgtattctacaggtagcaacgtgttccagacccgggccggctgcctgatcggcgccgagcacgtcaataatagctacgagtgcgacatccccattggagctggcatctgcgccagctaccagacgcaaacaaacagccctcgtagagcccggagcgtggccagtcagagtatcatcgcttacaccatgagcctgggcgccgaaaacagcgtggcctattctaacaatagcatcgccatccctaccaacttcaccatctctgtgacaaccgagattctgcctgtatccatgacaaagaccagtgtcgactgcactatgtacatctgcggcgatagcaccgaatgttctaacctcctgttgcagtatggaagcttttgtactcagctcaacagagccctgacaggcattgctgtggaacaggataagaacacccaggaggtgttcgcccaagtgaagcagatctataagacacctcctatcaaggacttcggcggcttcaactttagccagatcctgcctgacccgagcaagcctagcaaaaggtccttcatcgaggaccttctcttcaacaaggtgaccttggccgacgccggatttatcaagcagtacggcgactgcctgggagatatcgccgctagagatctgatttgtgctcaaaagttcaatggcctgaccgtgttaccgcctctgttgaccgatgagatgatcgcccagtacaccagcgccctgctggccggaacaatcaccagcggctggacattcggtgccggcgccgccctgcaaatccccttcgccatgcagatggcctaccggttcaatggtatcggcgtgacacaaaacgtgctgtacgagaaccagaaactgatcgccaaccagttcaacagcgccatcggaaagatccaggattctctgtcttctaccgccagtgccctgggcaagctgcaagacgtggtcaaccagaacgcccaggccctgaacacactggtgaaacagctgagctccaacttcggcgccatcagctccgtactgaatgacatcctgtcaagactggataaggtggaagccgaggtgcagattgatagactgatcaccggcagactgcagagcctgcagacatacgtgacccagcagctgatccgggctgctgagatcagagctagtgctaatctcgccgctacaaagatgagcgaatgcgtgctgggacagagcaagagagtggacttctgcggcaaggggtatcacctgatgtccttcccacagagcgcacctcacggcgtggtgtttttacatgtgacctacgtgcctgcccaggagaagaactttaccacggcccctgctatctgccacgatggcaaagcccacttccccagagagggcgtgttcgtaagcaacggcacacactggtttgtgacccagagaaacttctacgagccccagatcatcaccaccgacaacaccttcgtgtctggcaattgtgatgtggtgatcggcatcgtgaacaacaccgtgtacgaccctctgcagcccgagctggacagcttcaaagaggaactggacaagtacttcaagaaccacaccagccctgatgtggacctcggagacatcagcggcatcaacgcctccgtggtgaacatccagaaggaaattgacagactgaatgaggtggccaagaaccttaatgagagcctgatcgacctccaggagctgggcaagtacgagcagtacatcaagtggccctggtacatctggctggggttcattgcaggactgatcgccatcgtgatggtgaccatcatgctgtgctgcatgacctcttgttgtagctgcctgaagggctgctgctcctgcggcagctgttgcaagttcgacgaggatgacagcgagcctgtgctgaaaggcgtgaagctgcactacacctgaatcgat

SEQ ID NO: 16 - DNA, 252 bp, Artificial Sequence
  Synthetic- Codon optimized Envelope of SARS-CoV-2
gaattcaggaggtccaccatgtactcttttgtgagtgaagagacaggcaccctgatcgtgaatagcgtgctgttgtttctggccttcgtggtgttcctgctggtgaccctggctatcctgaccgccctgagactgtgcgcctactgctgcaacatcgtgaacgtgtccctggtcaagcccagcttctacgtgtacagcagagtgaagaacctgaacagcagccgggtgcccgacctgctggtgtgaatcgat

SEQ ID NO: 17 - DNA, 693 bp, Artificial Sequence
  Synthetic- Codon optimized Membrane of SARS-CoV-2
gaattcaggaggtccaccatggctgatagcaacggcacaatcaccgtggaagagctgaagaagctgcttgagcagtggaacctggtgattggatttctgtttctgacctggatctgcctgctgcagttcgcatacgccaaccggaacagattcctgtacatcatcaagctgatcttcctgtggctgctgtggcctgtgaccctggcctgcttcgtgctggccgccgtgtaccggatcaactggatcaccggcggaatcgccatcgccatggcctgtctggtgggcctgatgtggctgtcttatttcatcgcctcttttagactgttcgccagaaccagaagcatgtggtccttcaaccccgagacaaacatcctgcttaatgtgccactgcacggcaccatcctgacaagacctctgcttgaaagcgagctggtgatcggcgccgtgatcctgcggggccacctgagaatcgccggacaccacctgggcagatgcgacatcaaggacctgcctaaggaaatcaccgtggccaccagcagaacactgagctactacaagctcggcgcttctcagagagtggccggcgacagcggcttcgccgcttacagccggtacagaatcggcaactacaagctgaacaccgaccacagctccagcagcgacaacatcgccctgctggtccagtgaatcgat

SEQ ID NO: 18 - DNA, 1284 bp, Artificial Sequence
  Synthetic- Codon Optimized Nucleocapsid of SARS-CoV-2
gaattcaggaggtccaccatgtctgacaacggccctcaaaaccagcggaacgcccctcggataacatttggaggacccagcgacagcacaggctccaaccagaacggcgagagaagcggcgccagatctaaacagaggcggcctcagggcctgccaaacaacaccgccagctggttcaccgccctgacccaacacggcaaagaggacctcaagttccctagaggacagggcgtgcccatcaacacgaacagcagccccgacgaccagatcggctactacagacgggccacaagacggatcagaggcggcgacggcaagatgaaagacctgagcccaagatggtacttctactacctgggcaccggccccgaggccggcctgccctacggcgccaataaggacggcatcatctgggtcgccacagagggcgcactgaacacccccaaggaccacatcggaactagaaaccccgccaacaacgccgcaatcgtgctgcaactgccccaaggcacgaccctgcctaagggcttctacgccgaaggctctagaggcggcagccaggcttcttctcggagctcctcccggagccggaacagctctagaaattctacacctggcagcagcagaggcaccagccctgctagaatggccggcaacggcggagatgccgccctggccctgctgctgcttgatagactgaatcagctggaaagcaaaatgagcggaaaaggccagcaacagcaaggacaaacagtgacaaagaaaagcgccgctgaagcctctaagaaacctagacagaaaagaaccgccaccaaggcctacaacgtgacccaggcctttggcagaaggggacctgagcagacccagggaaatttcggcgatcaggagctgatccggcagggcaccgactacaagcactggcctcagatcgcccagttcgcccctagcgccagcgcttttttcggcatgagccggatcggcatggaagtgacccctagcggcacctggctgacatacaccggcgccatcaagctggacgataaggatcctaacttcaaggatcaggtgatcctgctgaacaagcatatcgacgcctataagaccttcccccccaccgagcccaagaaggacaagaagaaaaaggccgacgagacacaagctctgcctcagagacagaagaaacagcagaccgtgaccctgctgcctgccgccgacctggacgacttcagcaagcagctgcagcagagcatgagctctgctgatagcacccaggcctgaatcgat

SEQ ID NO: 19 - protein, 190 aa, Artificial Sequence
  Synthetic- RBD G339D
  MISC_FEATURE (19)..(19): G339D mutation
QPTESIVRFPNITNLCPFDEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 20 - protein, 190 aa, Artificial Sequence
  Synthetic- RBD S371L
  MISC_FEATURE (51)..(51): S371L mutation
QPTESIVRFPNITNLCPFGEVFNATRFASVYAWNRKRISNCVADYSVLYNLASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 21 - protein, 190 aa, Artificial Sequence
  Synthetic- RBD S373P
  MISC_FEATURE (53)..(53): S373P mutation
QPTESIVRFPNITNLCPFGEVFNATRFASVYAWNRKRISNCVADYSVLYNSAPFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 22 - protein, 190 aa, Artificial Sequence
  Synthetic- RBD S375F
  MISC_FEATURE (55)..(55): S375 mutation
QPTESIVRFPNITNLCPFGEVFNATRFASVYAWNRKRISNCVADYSVLYNSASFFTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 23 - protein, 190 aa, Artificial Sequence
  Synthetic- RBD K417N
  MISC_FEATURE (97)..(97): K417N mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 24 - protein, 190 aa, Artificial Sequence
  Synthetic- RBD K417T
  MISC_FEATURE (97)..(97): K417T mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 25 - protein, 190 aa, Artificial Sequence
  Synthetic- RBD K417M
  MISC_FEATURE (97)..(97): K417M mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGMIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 26 - protein, 190 aa, Artificial Sequence
  Synthetic- RBD N440K
  MISC_FEATURE (120)..(120): N440K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTNGVGYQPYRV

SEQ ID NO: 27 - protein, 190 aa, Artificial Sequence
  Synthetic- RBD G446S
  MISC_FEATURE (126)..(126): G446S mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVSGNYNYLYRLFRKSNLKPFERDISTEIYQAGSTPCNGVEGFNCYFPLQSYGFQPTNGVGYQPYRV

SEQ ID NO: 28 - protein, 190 aa, Artificial Sequence
  Synthetic- RBD L452R
  MISC_FEATURE (132)..(132): L452R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRV

SEQ ID NO: 29 - protein, 190 aa, Artificial Sequence
  Synthetic- RBD S477N
  MISC_FEATURE (157)..(157): S477N mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRV

SEQ ID NO: 30 - protein, 190 aa, Artificial Sequence
  Synthetic- RBD T478K
  MISC_FEATURE (158)..(158): T478K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSKPCNGVEGFNCYFPLQSYGFQPTNGVGYQPYRV

SEQ ID NO: 31 - protein, 190 aa, Artificial Sequence
  Synthetic- RBD E484K
  MISC_FEATURE (164)..(164): E484K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRV

SEQ ID NO: 32 - protein, 190 aa, Artificial Sequence
  Synthetic- RBD E484A
  MISC_FEATURE (164)..(164): E484A mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVAGFNCYFPLQSYGFQPTNGVGYQPYRV

SEQ ID NO: 33 - protein, 190 aa, Artificial Sequence
  Synthetic- RBD Q493K
  MISC_FEATURE (173)..(173): Q493K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLKSYGFQPTNGVGYQPYRV

SEQ ID NO: 34 - protein, 190 aa, Artificial Sequence
  Synthetic- RBD Q493R
  MISC_FEATURE (173)..(173): Q493R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLRSYGFQPTNGVGYQPYRV

SEQ ID NO: 35 - protein, 190 aa, Artificial Sequence
  Synthetic- RBD G496S
  MISC_FEATURE (176)..(176): G496S mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYSFQPTNGVGYQPYRV

SEQ ID NO: 36 - protein, 190 aa, Artificial Sequence
  Synthetic- RBD Q498R
  MISC_FEATURE (178)..(178): Q498R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFRPTNGVGYQPYRV

SEQ ID NO: 37 - protein, 190 aa, Artificial Sequence
  Synthetic- RBD N501Y
  MISC_FEATURE (181)..(181): N501Y mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRV

SEQ ID NO: 38 - protein, 190 aa, Artificial Sequence
  Synthetic- RBD Y505H
  MISC_FEATURE (185)..(185): Y505H mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGHQPYRV